(19) United States
(12) Patent Application Publication
(10) Pub. No.: US 2011/0071412 A1
(43) Pub. Date: Mar. 24, 2011

(54) Belt Type Bio-Signal Detecting Device

(75) Inventors: Bo-Jau KUO, Taipei (TW); Ching-Hsiu YANG, Taipei (TW)

(73) Assignee: NATIONAL YANG MING UNIVERSITY, Taipei (TW)

(21) Appl. No.: 12/624,265

(22) Filed: Nov. 23, 2009

(30) Foreign Application Priority Data
Sep. 18, 2009 (TW) 098131508

(51) Int. Cl. A61B 5/0408

(52) U.S. Cl. 600/509

(57) ABSTRACT

This invention relates to a bio-signal detecting device which can be belted up on a user. The bio-signal detecting device includes a respiratory signal detecting module, a electrocardiogram (ECG) signal detecting module, a body temperature detecting module, a contacting object and a flexible belt, wherein the respiration signal detecting module is used for obtaining a respiratory signal, the ECG signal detecting module is used for obtaining a ECG signal and the body temperature detecting module is used for a temperature value. The respiratory signal detecting module, the ECG signal detecting module, and the body temperature detecting module are disposed on the contacting object. The flexible belt is coupled to the contacting object and belts the user in order to fix the contacting object on the user.

### BACKGROUND OF THE INVENTION

1. Field of the invention

The present invention relates to a bio-signal detecting device, and more particularly, to a bio-signal detecting device that detects many kinds of bio-signals.

2. Prior art

Conventional electrocardiogram detection is difficult. Wet electrodes within electrolyte are often used as the interface between skin and detecting machine during electrocardiogram detection. However, the electrodes must stick to the subject's skin and cause minor damage to the skin when tearing off after each usage.

Nevertheless, the electrode stickers are disposable, which increases the cost and might cause allergy and infection on subject's skin during long-term detection. Those disadvantages bring about some impact on the popularity of the electrocardiogram detection.

Besides, elderly care is more important when the number of elders is increased. Generally speaking, in order to determine whether a man is healthy, we often need to detect the subject's bio-signals such as body temperature. Although there are various kinds of home care detecting devices, those devices are often used when necessary, not to be used to monitor the elders' physiological status at any time. Moreover, many elderly people live alone, even if they use those detecting devices to obtain various kinds of bio-signals, doctors and nurses at a remote end can not know the physiological status of elderly people immediately.

### SUMMARY OF THE INVENTION

One object of the present invention is to provide a bio-signal detecting device to improve the defects of conventional use.

According to the object of the present invention, the bio-signal detecting device is to be worn on the subject. The bio-signal detecting device includes an ECG signal detecting module, a contacting object and a flexible belt. The ECG signal detecting module is disposed on the contacting object, and the ECG signal detecting module has a plurality of dry electrode contact portion. The flexible belt couples to the contacting object and winds around the subject's body for the ECG signal detecting module to obtain an ECG signal from the dry electrode contact portion.

In one embodiment of the present invention, the flexible belt can be wound around the contacting object by coiling to achieve coupling effect. Furthermore, the bio-signal detecting device provided in the present embodiment can also integrate with respiratory detecting module and body temperature detecting module to obtain more bio-signals.

The advantage of the present invention is to provide a useful, convenient, stable, comfortable detecting device for continuous monitoring with alert function. It can be used to detect three important physiological parameters such as the ECG signals, respiration signals and the body temperature at the same time. Thus, it can be worn for long term and detect bio signals anytime as an early warning system. The present invention achieves the best and the most convenient way for recording the electrocardiogram, respiration and body temperature signals, and increases the variety of applications of bio-signals.

The foregoing aspects and many of the attendant advantages of this invention will become more readily appreciated as the same becomes better understood by reference to the following detailed description, when taken in conjunction with the accompanying drawings.

### BRIEF DESCRIPTION OF THE DRAWINGS

FIG. 1 illustrates the bio-signal detecting device of an exemplary according to the preferred embodiment of the present invention that is worn on a subject's body;

FIG. 2 illustrates the functional block diagram of an exemplary of the bio-signal detecting device in a preferred embodiment of the present invention;

FIG. 3A illustrates a chart of an exemplary that shows the first model of the bio-signal detecting device in a preferred embodiment of the present invention;

FIG. 3B illustrates a top view of an exemplary of the bio-signal detecting device in a preferred embodiment of the present invention.

### DETAILED DESCRIPTION OF THE PREFERRED EMBODIMENT

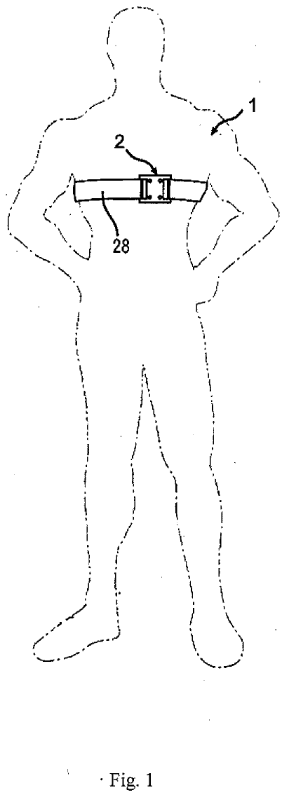

FIG. 1 shows the bio-signal detecting device worn on a subject's body according to the preferred embodiment of the present invention. The bio-signal detecting device 2 provided in the preferred embodiment of the present invention is suitable for fixing on the body of subject 1 with a flexible belt 28. The bio-signal detecting device 2 is belted between the chest and the abdomen of the subject 1 by the flexible belt 28 in the present embodiment. In the preferred embodiment, the material of the flexible belt 28 can be leather, cotton, or metal as long as the flexible belt 28 possesses flexibility.

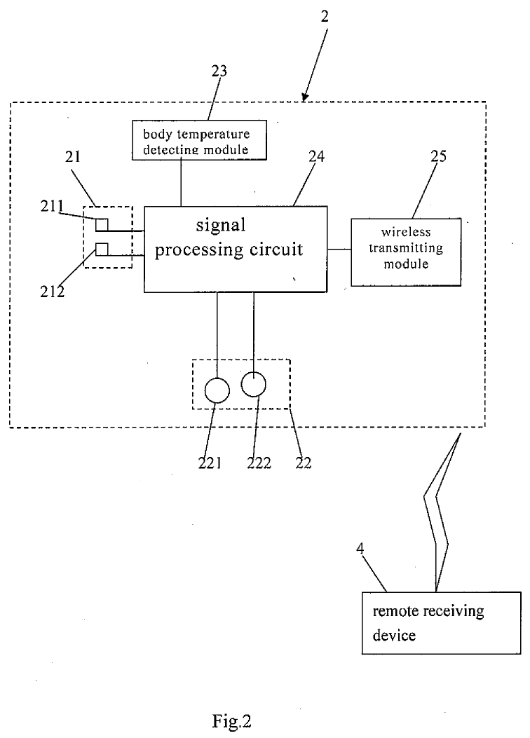

FIG. 2 is the functional block diagram of the bio-signal detecting device in a preferred embodiment of the present invention. In FIG. 2, the bio-signal detecting device 2 includes an ECG signal detecting module 21, a respiratory signal detecting module 22, a body temperature detecting module 23, a signal processing circuit 24 and a wireless transmitting module 25. The ECG signal detecting 21 includes a plurality of dry electrode contacting portion 211, 212, and the respiratory signal detecting module 22 comprises a plurality of force detecting unit 221, 222.

The ECG signal detecting module 21 is to obtain the ECG signals, the respiratory signal detecting module 22 is to obtain the respiratory signals, and the body temperature detecting module 23 is to obtain the body temperature signals.

The signal processing circuit 24 couples to the ECG signal detecting module 21, the respiratory signal detecting module 22, the body temperature detecting module 23, and the wireless transmitting module 25 respectively. In the present embodiment, the signal processing circuit 24 comprises a filter circuit, an amplifier circuit, an Analog-to-Digital circuit, a digital signal processing circuit and a micro processor to process the bio-signals obtained from the ECG signal detecting module 21, the respiratory signal detecting module 22, and the body temperature detecting module 23. The signal processing circuit 24 can transmit the processed bio-signals to a remote receiving device 4 in a distant location via the wireless transmitting module 25.

In the present embodiment, the remote receiving device 4 can be any tools that receive and analyze signals, such as a mobile phone, a PDA, a MP3, a watch, a blood pressure monitor, a blood sugar meter, a thermometer, an ear thermometer, even an activity detector or other specific instruments. These instruments can transmit the analyzed data to a monitoring device of a family doctor in a predetermined time or real-time after receiving and analyzing the received signals. In some embodiments, the remote receiving device 4 is the monitoring device of a family doctor.

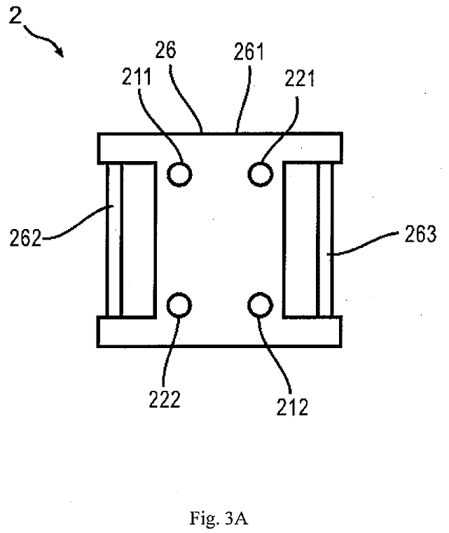

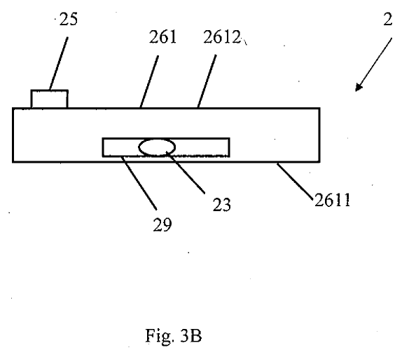

FIG. 3A is a chart that shows the first model of the bio-signal detecting device in a preferred embodiment of the present invention. FIG. 3B shows the top view of the bio-signal detecting device in a preferred embodiment of the present invention. Detail description of the first preferred embodiment will conduct with FIG. 1, FIG. 2, FIG. 3A and FIG. 3B.

The bio-signal detecting device 2 comprises a contacting object 26 and a flexible belt 28. The contacting object 26 further includes a body 261 and two supporters 262, 263. The body 261 has a first surface 2611 and a second surface 2612. In the present embodiment, two supporters 262, 263 are made of metal. The ECG signal detecting module 21, the respiratory signal detecting module 22, the body temperature detecting module 23, the signal processing circuit 24 and the wireless transmitting module 25 described above are all disposed in the body 261.

In the present embodiment, the body temperature detecting module 23 is a thermistor that can be disposed on the first surface 2611 of the body 261. In the present embodiment, a thin metal sheet 29 is attached to the first surface 2611 of the body 261. The thin metal sheet 29 is flat and can be attached to a subject closely for a better contact. The temperature is balanced in the thin metal sheet 29. The other side of the thin metal sheet 29 contacts the body temperature detecting module 23 and transfers the temperature of the subject 1 into voltage signal to obtain the temperature signal. And the temperature signal is transmitted to the signal processing circuit 24 for further processing.

The wireless transmitting module 25 described above is disposed on the second surface 2612 of the body 261. In other embodiments, the wireless transmitting module 25 can be disposed in the body 261 without any limitation.

In the embodiment, two supporters 262, 263 described above are disposed on two sides of the body 261 and form a duel retaining rings. In the present embodiment, the dry electrode contacting portions 211, 212 described above couple the metal supporters 262, 263 to contact the skin of the subject 1 indirectly to obtain the ECG signal via metal supporters 262, 263. In other embodiments, dry electrode contacting portions 211, 212 can also be disposed on the first surface 2611 of the body 261 for contacting the skin of the subject 1 directly to obtain ECG signal.

The flexible belt 28 repstively winds around two supporters 262, 263 (duel retaining rings) to couple the contacting object 26. Furthermore, the flexible belt 28 can also winds around the body of the subject 1 (ex. wind around the body of the user for one lap), so that the dry electrode contacting portions 211, 212 of the ECG signal detecting module 21 contact the skin of the subject 1 through metal supporters 262, 263 for obtaining ECG signals. In the present embodiment, the ECG signals include electrocardiogram signals. In other embodiments, the ECG signals also comprise the electrocardiogram signals and heart rate variability signals.

In the present embodiment, the plurality of the dry electrode contacting portions 211, 212 of the ECG signal detecting module 21 are disposed on the upper left and lower right of the body 261 of the contacting object 26. The force detecting units 221, 222 of the respiratory signal detecting module 22 are disposed on the upper right and the lower left of the body 261 of the contacting object 26. It is not limited for the location of the ECG signal detecting module 21 and the respiratory signal detecting module 22 in the present invention.

In the present embodiment, the plurality of the dry electrode contacting portions 211, 212 of the ECG signal detecting module 21 are disposed in different locations on the body 261 of the contacting object 26 respectively, to collect the electric signals from the metal supporters 262, 263 by contacting skin. Moreover, the dry electrode contacting portions 211, 212 are disposed on the left side and the right side of the body 261 so that the ECG signal from two sides of the body 261 are collected. Furthermore, the body temperature detecting module 23 is the reference for the ECG signal detecting module to generate the ECG signal. The body temperature detecting module 23 and the dry electrode contacting portions 211, 212 configure a three-point electrocardiogram input structure to collect better ECG signals.

Referring to FIG. 1, FIG. 2 and FIG. 3A, the flexible belt 28 winds around two metal supporters 262, 263 of the retaining ring in the present embodiment. After the flexible belt 28 winds around the body of the subject 1 for one lap, the flexible belt 28 produces a little tensile force to drive the force detecting unit 221, 222, and thus generates the respiratory signal.

Therefore, the change of chest expansion of the subject 1 while breathing will cause the change of tensile force of the flexible belt 28. The change of tensile force will be detected by the force detecting unit 221, 222, thus the continuous upper (chest) and lower (abdomen) respiratory signals are obtained.

In conclusion, the bio-signal detecting device provided in the preferred embodiment in the present invention detects various bio-signals. The detecting device is configured alike the retaining ring of the belt, and the dimension is alike the retaining ring of a belt as well. The detecting device winds around the subject between the chest and the abdomen by the flexible belt to detect three important physiological parameters such as the ECG signal, respiratory signal and the body temperature. Thus, it can be worn for long term and detect bio signals anytime as an early waning system. The present invention achieves the best and the most convenient way for recording the electrocardiogram, respiratory and body temperature signals, and increases the variety of applications of bio-signals.

While the preferred embodiment of the invention has been illustrated and described, it will be appreciated that various changes can be made therein without departing from the spirit and scope of the invention.

What is claimed is:

1. A bio-signal detecting device which can be belted up on a subject, the bio-signal detecting device comprising:
a contacting object;
an electrocardiogram (ECG) signal detecting module disposed on the contacting object, and the ECG signal detecting module has a plurality of dry electrode contact portion; and
a flexible belt coupled to the contacting object, and wound around the subject's body to obtain an ECG signal via the dry electrode contact portion.

2. The bio-signal detecting device as claimed in claim 1, which further comprises a respiratory signal detecting module disposed on the contacting object for obtaining a respiration signal.

3. The bio-signal detecting device as claimed in claim 1, wherein the ECG signal comprises an electrocardiogram signal and a heart rate variability signal.

4. The bio-signal detecting device as claimed in claim 1, which further comprises a body temperature detecting module for obtaining a temperature value, the contacting object has a first surface, and the body temperature detecting module is disposed on the first surface and attached on the subject.

5. The bio-signal detecting device as claimed in claim 4, which further comprises a wireless transmitting module disposed on the contacting object, and electronically connected to the body temperature detecting module to transmit the temperature signal to a remote receiving device.

6. The bio-signal detecting device as claimed in claim 4, wherein the body temperature detecting module is the reference for the ECG signal detecting module to generate the ECG signals, the body temperature detecting module and the dry electrode contacting portions configure a three-point electrocardiogram input structure.

7. The bio-signal detecting device as claimed in claim 1, which further comprises a respiratory signal detecting module and a wireless transmitting module, the respiratory signal detecting module and the wireless transmitting module are disposed on the contacting object, the respiratory signal detecting module is used for obtaining a respiratory signal, and the wireless transmitting module is electrically connected to the respiratory signal detecting module and the ECG signal detecting module to transmit the respiratory signal and the ECG signal to a remote receiving device.

8. The bio-signal detecting device as claimed in claim 7, wherein the contacting object has a second surface, and the wireless transmitting module is disposed on the second surface.

9. The bio-signal detecting device as claimed in claim 2, wherein the contacting object further comprises a body and two supporters, the supporters are disposed on two sides of the body, and the flexible belt winds around the supporters and the body of the subject respectively.

10. The bio-signal detecting device as claimed in claim 9, wherein the dry electrode contacting portions are coupled to the supporters, the supporters contact the skin of the subject, and the dry electrode contacting portions obtain the ECG signal via the supporters.

11. The bio-signal detecting device as claimed in claim 9, wherein the respiratory signal detecting module further comprises two force detecting units, the flexible belt winds around the supporters and the body of the subject, and a tensile force is produced, the tensile force drives the force detecting unit and generates the respiratory signal.

12. The bio-signal detecting device as claimed in claim 1, wherein the contacting object is an object with duel retaining rings, the flexible belt couples to the duel retaining rings respectively.

13. The bio-signal detecting device as claimed in claim 1, wherein the flexible belt is made of leather, cotton or metal.

14. The bio-signal detecting device as claimed in claim 1, wherein the flexible belt winds around the body of the subject between the chest and the abdomen.
